(19) United States
(12) Patent Application Publication
(10) Pub. No.: US 2018/0271788 A1
(43) Pub. Date: Sep. 27, 2018

(54) VESICLE CONTAINING METALLIC NANOPARTICLE AND METHOD FOR PRODUCTION THEREOF

(71) Applicant: The United States of America, as represented by the Secretary, Department of Health and Human Serv, Bethesda, MD (US)

(72) Inventors: Xiaoyuan Chen, Potomac, MD (US); Jibin Song, Bethesda, MD (US)

(73) Assignees: The United States of America, as represented by the Secretary, Department of Health and; Human Services, Bethesda, MD (US)

(21) Appl. No.: 15/781,940

(22) PCT Filed: Dec. 9, 2016
(86) PCT No.: PCT/US2016/065708

§ 371 Date: Jun. 6, 2018

Related U.S. Application Data
(60) Provisional application No. 62/266,289, filed on Dec. 11, 2015.

(51) Int. Cl. A61K 9/127; A61K 51/12; A61K 41/00; A61P 35/00; A61K 49/22; B01J 13/12; A61N 5/06

(52) U.S. Cl. CPC A61K 9/1272; A61K 9/1278; A61K 51/1237; A61N 5/0625; A61P 35/00; A61K 49/227; B01J 13/12; A61K 41/0052

(57) ABSTRACT

Disclosed is a method of producing a vesicle containing a metallic nanoparticle that is covalently bound to at least one hydrophilic polymer and at least one hydrophobic polymer, wherein the method involves dispersing the polymer-bound metallic nanoparticle in an organic solvent, adding an aqueous solution containing a dispersing aid to form a mixed solution, sonicating the mixed solution to form an emulsion; and removing the organic solvent from the emulsion until the vesicle forms. Using this method, the formed vesicle has a diameter of 20-150 nm, which is useful for a method of conducting photothermal therapy (PTT) for killing cells, such as cancer cells.

### CROSS-REFERENCE TO A RELATED APPLICATION

This patent application claims the benefit of U.S. Provisional Patent Application No. 62/266,289, filed Dec. 11, 2015, which is incorporated by reference.

### BACKGROUND OF THE INVENTION

Plasmonic nanostructures, such as gold nanorods (AuNRs), nanoparticles, nanocages, and nanoshells have been actively studied as cancer theranostics. See, for example, Giljohann et al., Angew. Chem. Int. Ed. 2010, 49, 3280-3294; Ament et al., Nano Lett. 2012, 12, 1092-1095; Zhang et al., J. Am. Chem. Soc. 2014, 136, 7317-7326; Lozano et al., J. Am. Chem. Soc. 2012, 134, 13256-13258; Yuan et al., Angew. Chem. Int. Ed. 2013, 52, 13965-13969; Huang et al., J. Am. Chem. Soc. 2006, 128, 2115-2120; von Maltzahn et al., Cancer Res. 2009, 69, 3892-3900; and Mallidi et al., Trends Biotechnol. 2011, 29, 213-221. Due to the presence of tunable localized surface plasmon resonance (LSPR), plasmonic nanostructures not only serve as attractive probes for cancer imaging but also act as highly localized heat sources when irradiated with a laser through the photothermal effect (Choi et al., ACS Nano 2011, 1995-2003; Giljohann et al., Angew. Chem. Int. Ed. 2010, 49, 3280-3294; and Gao et al., Nanoscale 2013, 5, 5677-5691). Plasmonic coupling between gold nanocrystals generates enhanced electromagnetic field, leading to increased photothermal conversion efficiency and optical properties, such as enhanced scattering light and photoacoustic (PA) signal (Aslan et al., Curr. Opin. Chem. Biol. 2005, 9, 538-544; Nie et al., Chem. Soc. Rev. 2014, 43, 7132-7170; Li et al., Nanomedicine 2015, 10, 299-320; Huang et al., Angew. Chem. Int. Ed. 2013, 52, 13958-13964; and Halas et al., Chem. Rev. 2011, 111, 3913-3961).

There have been reports of theranostic platforms for real-time diagnosis and cancer therapy (Anker et al., Nat. Mater. 2008, 7, 442-453; Tam et al., ACS Nano 2010, 4, 2178-2184; and Yan et al., ACS Nano 2012, 6, 3663-3669). Plasmonic vesicles with doxorubicin (DOX) loaded into the hollow cavity have been shown to be delivered into cancer cells, and this combination leads to simultaneous localized chemotherapy and thermal therapy in a near infrared (NIR) laser responsive manner (Song et al., ACS Nano 2013, 7, 9947-9960). However, the relatively large size of these vesicles (>200 nm) can only be administered locally since intravenous injection would cause rapid accumulation in the reticuloendothelial system (RES) organs and tissues, such as the liver and spleen. Even after the vesicles were degraded over time, the individual AuNRs with a width greater than 8 nm and length of about 40 nm were not readily excreted from the body (Xu et al., J. Am. Chem. Soc. 2011, 134, 1699-1709; Wang et al., Nano Lett. 2010, 11, 772-780; von Maltzahn et al., Cancer Res. 2009, 69, 3892-3900; Sun et al., ACS Nano 2014, 8 8438-8446; and Zhang et al., Adv. Mater. 2012, 24, 1418-1423).

Thus, there remains a need to provide plasmonic assemblies with high accumulation efficiency that are suitable for diagnostic and therapeutic uses and that rapidly clear from the body after administration.

### BRIEF SUMMARY OF THE INVENTION

The invention provides a method of producing a vesicle comprising a metallic nanoparticle that is covalently bound to at least one hydrophilic polymer and at least one hydrophobic polymer, wherein the method comprises

dispersing the polymer-bound metallic nanoparticle in an organic solvent,

adding an aqueous solution comprising a dispersing aid to form a mixture,

sonicating the mixture to form an emulsion; and

removing the organic solvent from the emulsion until the vesicle forms, wherein

the polymer-bound metallic nanoparticle comprises a metallic nanoparticle that is covalently bound to at least one hydrophilic polymer and at least one hydrophobic polymer, and

the vesicle has a diameter of 20-150 nm.

Also provided is a vesicle comprising a polymer-bound metallic nanoparticle comprising a metallic nanoparticle that is covalently bound to at least one hydrophilic polymer and at least one hydrophobic polymer, wherein the vesicle has a diameter of 20-150 nm.

### BRIEF DESCRIPTION OF THE SEVERAL VIEWS OF THE DRAWING(S)

FIG. 1 is a schematic illustration of the preparation of small AuNR vesicles assembled from small gold nanorods (AuNRs) coated with poly(ethylene glycol) (PEG) and poly(lactic-co-glycolic acid) (PLGA) using an oil-in-water (O/W) emulsion method.

FIG. 2 depicts UV-Vis spectra of AuNR@PEG/PLGA in chloroform and AuNR vesicles with different sizes (60 nm, 80 nm, and 96 nm) in water.

FIG. 3 is an ¹H NMR spectra (300 MHz, 6, ppm, D₂O) of AuNR@PEG/PLGA vesicle after incubation in cell culture medium at day 0 (a) and day 10 (b). The NMR (nuclear magnetic resonance) results of the vesicle after being incubated in cell culture medium for 10 days confirmed the degradation of PLGA by the appearance of CH₃— of oligo(lactic acid) (OLA) and lactic acid (LA), and the CH₂— of glycolic acid (GA) and CH— of LA new peaks. The PLGA was almost completely degraded, and the vesicle was disassociated into AuNR mainly coated with PEG.

FIG. 4 is a graph demonstrating the viability of the cells incubated with small AuNR@PEG or AuNR vesicles without and with different power density of 808 nm laser for 5 min irradiation.

FIG. 5 is a bar graph demonstrating the cell viability in the presence of AuNR vesicles at different concentrations: 0.25 nM (first bar), 0.5 nM (second bar), 1 nM (third bar) and 2 nM (fourth bar) after an incubation for 2 h, 4 h, 8 h, 16 h, and 24 h.

FIG. 6 is a plot of the photoacoustic (PA) signal of a tumor treated with small AuNR@PEG or AuNR@PEG/PLGA vesicles at different time points post-injection.

FIG. 7 is a plot illustrating the blood clearance of [⁶⁴Cu]—AuNR vesicles (injected dose (ID) per gram (g)) in mice over time (hours).

FIG. 8 is a bar graph of the quantitative region of interest (ROI) analysis of tumor, muscle, and liver at 2 h (first bar), 6 h (second bar), 24 h (third bar), and 48 h (fourth bar) post-injection of 150 μCi of [⁶⁴Cu]—Au@PEG/PLGA vesicles (injected dose (ID) per gram (g)).

FIG. 9 is a bar graph of the biodistribution of AuNR vesicles in mice bearing tumors at day 1 (first bar) and day 10 (second bar) post-injection measured by inductively coupled plasma mass spectrometric (ICP-MS) analysis of Au in different organs and tissues (injected dose (ID) per gram (g)).

FIG. 10 is a bar graph of the quantitative region of interest (ROI) analysis of tumor, muscle and liver at 2 h (first bar), 6 h (second bar), 24 h (third bar), and 48 h (fourth bar) post-injection of 150 μCi of [⁶⁴Cu]AuNR@PEG in a control experiment (injected dose (ID) per gram (g)).

FIG. 11 is a bar graph of the biodistribution of AuNR@PEG/PS vesicles in mice bearing tumors at day 1 (first bar) and day 10 (second bar) post-injection measured by inductively coupled plasma mass spectrometric (ICP-MS) analysis of Au in different organs and tissues.

FIG. 12 is a graph illustrating the temperature changes of the tumor region treated with small AuNRs and AuNR vesicles and irradiated with 808 nm laser at different power densities.

FIG. 13 is a graph of tumor growth curves of the relative tumor volume (V/Vₒ) over time (days), in which ▪ is a control; ● is PBS+0.8 W/cm²; ▴ is AuNR vesicles; ▾ is AuNR+0.8 W/cm; and [symbol] is AuNR vesicles+0.8 W/cm².

FIG. 14 is graph of survival curves of tumor-bearing mice treated with PBS with laser irradiation (●), small AuNRs with laser irradiation (▾), and AuNR vesicles with ( [symbol] ) and without (▴) and laser irradiation relative to a control (▪).

### DETAILED DESCRIPTION OF THE INVENTION

The invention provides a method of synthesizing ultrasmall, dissociable plasmonic vesicles that are able to provide one or more of the following features: prolonged circulation, tumor accumulation, rapid excretion from the body, enhanced photoacoustic signal, enhanced photothermal effect, and/or high photothermal cancer therapy efficacy. To this end, the invention provides a method of producing a vesicle comprising a metallic nanoparticle that is covalently bound to at least one hydrophilic polymer and at least one hydrophobic polymer, wherein the method comprises

dispersing the polymer-bound metallic nanoparticle in an organic solvent,

adding an aqueous solution comprising a dispersing aid to form a mixture,

sonicating the mixture to form an emulsion; and

removing the organic solvent from the emulsion until the vesicle forms, wherein

the polymer-bound metallic nanoparticle comprises a metallic nanoparticle that is covalently bound to at least one hydrophilic polymer and at least one hydrophobic polymer, and

the vesicle has a diameter of 20-150 nm.

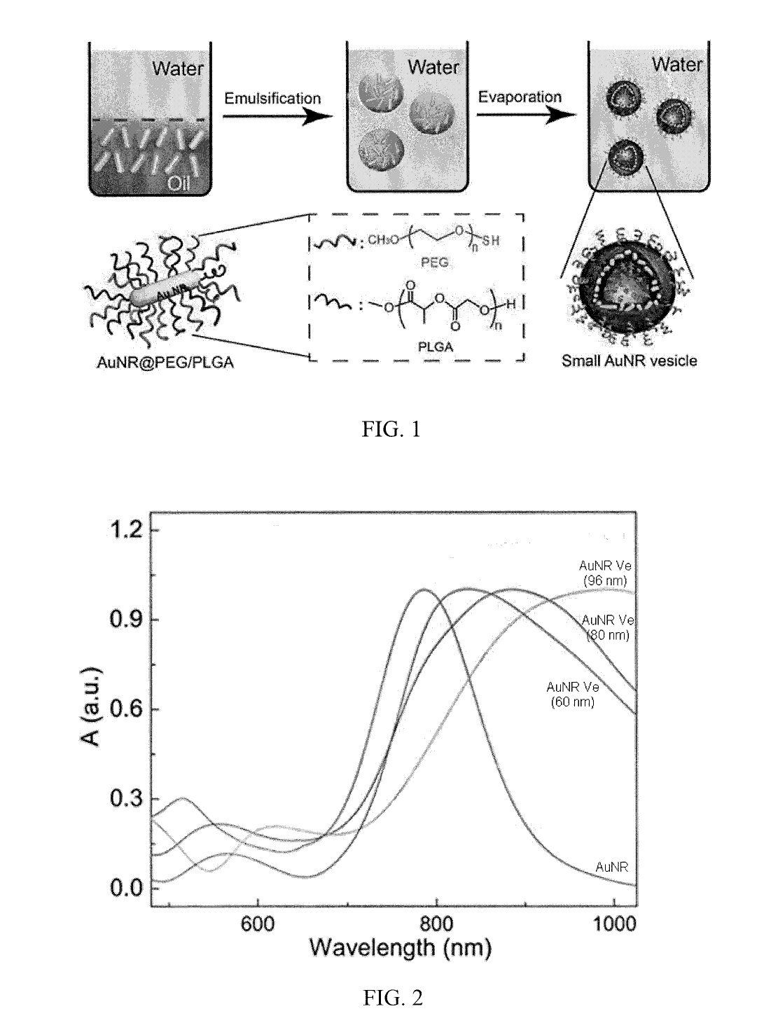

In a specific example of this method, FIG. 1 illustrates the preparation of small gold nanorod vesicles assembled from small gold nanorods (AuNRs) coated (e.g., grafted) with poly(ethylene glycol) (PEG) and poly(lactic-co-glycolic acid) (PLGA) using the oil-in-water (O/W) emulsion method described above.

Using this method, the amphiphilic metallic nanoparticles that are coated (e.g., grafted) with the hydrophilic brush polymer and hydrophobic brush polymer self-assemble into plasmonic vesicles with the metallic nanoparticles embedded in the shell formed by the hydrophobic polymer chains and the hydrophilic polymer chains extend to both sides of the vesicle, which serves to stabilize the structure and can enable further biomedical applications due to its excellent protein resistant properties.

Accordingly, the invention further provides a vesicle comprising a polymer-bound metallic nanoparticle comprising a metallic nanoparticle that is covalently bound to at least one hydrophilic polymer and at least one hydrophobic polymer, wherein the vesicle has a diameter of 20-150 nm. In certain embodiments, the vesicle is prepared by the inventive method set forth herein.

The metallic nanoparticle comprises any metal that is biocompatible and nontoxic. For example, the metal can be gold, iron oxide, copper disulfide silver, nickel, cobalt, platinum, palladium, iridium, or mixtures thereof. Preferably, the metallic nanoparticle comprises gold.

The metallic nanoparticle can be in any suitable size and shape that can be used to form a vesicle. For example, the size of the nanoparticle will be on the nanoscale, such that no dimension of the nanoparticle is larger than about 30 nm. In any of the embodiments described herein, the dimensions of the nanoparticle (e.g., the diameter, width, length, and/or height) is less than 25 nm (e.g., less than 20 nm, less than 18 nm, less than 15 nm, less than 12 nm, less than 10 nm, less than 8 nm, less than 7 nm, less than 6 nm, less than 5 nm, less than 4 nm, less than 3 nm, less than 2 nm, or less than 1 nm). Any two of the foregoing values can be used as an endpoint to define a close-ended range, or can be used singly to define an open-ended range. For example, the nanoparticle can have a diameter of less than about 30 nm or less than about 20 nm or the diameter can have a length and/or width of less than about 30 nm or less than about 20 nm.

Typically, the nanoparticle will be in the shape of a sphere (nanosphere) or a rod (nanorod). In some embodiments, the metallic nanoparticle is a quantum dot, which is a particle made from semiconducting materials and that fluoresces in the visible range. The quantum dot can be made from a single material (e.g., CdS, CdSe, ZnS, or ZnSe) or multiple materials in the form of an alloy (e.g., CdSₓSe₁₋ₓ/ZnS) or a core-shell structure (e.g., CdSe core with a ZnS shell).

Preferably, the metallic nanoparticle is a quantum dot or nanorod. In a specific example, a nanorod is used that is about 8 nm long and about 2 nm wide.

The formed vesicles desirably are small, i.e., less than 200 nm in diameter, in order to improve the in vivo clearance from a subject. Typically, the vesicles will have a diameter that ranges from 20-150 nm (e.g., 50-125 nm, 60-100 nm, 60-90 nm). For example, the diameter can be at least 20 nm (e.g., at least 30 nm, at least 40 nm, at least 50 nm, at least 55 nm, at least 60 nm, at least 65 nm, at least 70 nm, at least 75 nm) and is less than 200 nm (e.g., less than 180 nm, less than 170 nm, less than 150 nm, less than 125 nm, less than 110 nm, less than 100 nm, less than 99 nm, less than 98 nm, less than 95 nm, less than 90 nm, less than 85 nm, less than 80 nm). Any two of the foregoing endpoints can be used to define a close-ended range, or can be used singly to define an open-ended range.

The hydrophilic polymer is any polymer that is soluble in or swollen by water and typically includes one or more polar or charged functional groups (e.g., hydroxyl, carboxy, cyano, ether, imino, acrylamide). In any of the embodiments, the hydrophilic polymer is at least one polymer selected from polyethylene glycol (PEG), poly(vinyl alcohol) (PVA), poly(vinylpyrrolidone) (PVP), polyacrylic acid, poly(meth)acrylic acid, polyethylenimine (PEI), poly(methyl vinyl ether), poly(styrene-maleic acid), polyethylene glycol ether, polyamide, polyacrylamide, a polypeptide, and a DNA. If desired, more than one type of hydrophilic polymer can be used in combination. In some embodiments, the hydrophilic polymer comprises polyethylene glycol (PEG), poly(vinyl alcohol) (PVA), poly(vinylpyrrolidone) (PVP), polyacrylic acid, poly(meth)acrylic acid, polyethylenimine (PEI), or a combination thereof. Preferably, the hydrophilic polymer comprises polyethylene glycol (PEG).

The hydrophobic polymer is any polymer that is sparingly soluble in water (e.g., the macroscopic surface of the polymer is not wetted by water) and typically includes very few or no polar or charged functional groups (e.g., hydroxyl, carboxy, cyano, ether, imino, acrylamide). The polymer can contain, for example, one or more types of pendant groups, such as alkyl, aryl, and haloalkyl. In any of the embodiments, the hydrophobic polymer comprises at least one polymer selected from poly(lactic-glycoacid) (PLGA), polylactide (PLA), polystyrene, polyethylene, polypropylene, poly(2-dimethylaminoethylmethacrylate) (PDMAEMA), poly(N-isopropylacrylamide) (PNIPAM), polybutadiene, polyisoprene, poly(styrene-butadiene), polyvinyl chloride, polytetrafluoroethylene, polydimethylsiloxane, polycaprolactone, poly(4-vinylpyridine), poly(ethyl acrylate), poly(methyl acrylate), and poly(methyl methacrylate) (PMMA). If desired, more than one type of hydrophobic polymer can be used in combination. In some embodiments, the hydrophobic polymer comprises poly(lactic-glycoacid) (PLGA), polylactide (PLA), or both.

The degree of hydrophilicity and hydrophobicity can be measured by any suitable method, such as a water contact angle measurement, which quantifies the wettability of a surface by a liquid. For example, a thick film of the polymer sample is provided on a clean substrate (e.g., glass slide), and a drop of water is added to the polymer surface. A BET instrument can then be used to estimate the surface tension of the polymer by measuring the contact angle (e.g., using Young's equation). The higher the contact angle (>90°, such as greater than 90° and up to 1800, greater than 90° and up to 1500, greater than 90° and up to 140°, or greater than 90° and up to 120°), the poorer the wettability and the greater the hydrophobicity of the polymer. Conversely, the lower the contact angle (0-90°), the better the wettability and the greater the hydrophilicity of the polymer. In a specific embodiment, a contact angle and sliding angle can be measured for a particular polymer sample with the DataPhysics Optical Contact Angle (OCA) measurement device (Filderstadt, Germany).

The hydrophilic and hydrophobic polymers can have any suitable average molecular weight, which typically is tuned based on the desired solubility properties and/or end use. For example, the number, weight, or volume average molecular weight can be at least about 200 g/mol (e.g., at least about 300 g/mol, at least about 500 g/mol, at least about 800 g/mol, at least about 1,000 g/mol, at least about 1,500 g/mol, at least about 2,000 g/mol, at least about 3,000 g/mol, at least about 4,000 g/mol, at least about 5,000 g/mol, at least about 6,000 g/mol, at least about 8,000 g/mol) and/or up to about 100,000 g/mol (e.g., up to about 90,000 g/mol, or up to about 80,000 g/mol, up to about 70,000 g/mol, up to about 60,000 g/mol, up to about 50,000 g/mol, up to about 40,000 g/mol, up to about 30,000 g/mol, up to about 20,000 g/mol, up to about 10,000 g/mol, up to about 8,000 g/mol, or up to about 6,000 g/mol). These lower and upper limits with respect to the number, weight, or volume average molecular weight can be used in any combination to describe the polymer molecular weight range (e.g., about 200 to about 100,000 g/mol, about 300 g/mol to about 50,000 g/mol, and about 1,000 to about 20,000 g/mol, etc.). In any of the embodiments described herein, the molecular weight of the hydrophilic polymer ranges from about 1,000 g/mol to about 15,000 g/mol (e.g., from about 2,000 g/mol to about 10,000 g/mol). In any of the embodiments described herein, the molecular weight of the hydrophobic polymer ranges from about 10,000 g/mol to about 50,000 g/mol (e.g., from about 15,000 g/mol to about 35,000 g/mol).

The hydrophilic and hydrophobic polymers can be characterized quantitatively using known methods. For example, molecular weight determinations can be made using gel permeation chromatography (also known as size exclusion chromatography and gel filtration chromatography), nuclear magnetic resonance spectroscopy (NMR) (e.g., ¹H, ¹³C), matrix-assisted laser desorption/ionization mass spectroscopy (MALDI), light scattering (e.g., low angle and multi angle), matrix assisted laser desorption ionization time of flight (MALDI-TOF) mass spectrometry, MALDI-TOF MS coupled with collision induced dissociation (CID), small angle neutron scattering (SANS), sedimentation velocity, end group analysis, osmometry, cryoscopy/ebulliometry, and viscometry.

The graft density is the number of polymer chains that occupy an area of the metallic nanoparticle (e.g., quantum dot, nanosphere, or nanorod). The degree of graft density typically is determined based on the desired end use. With a high graft density, the polymer chains tend to form a brush-like structure. In general, a higher concentration of polymer and/or a longer contact time will provide a higher graft density on the metallic nanoparticle surface. For example, the graft density typically can range from about 0.1 to 1 chains/nm² (e.g., about 0.1 to 0.8 chains/nm², about 0.1 to 0.6 chains/nm², about 0.1 to 0.5 chains/nm², about 0.1 to 0.4 chains/nm², about 0.2 to 0.8 chains/nm², about 0.3 to 0.6 chains/nm², about 0.4 chains/nm²). In addition, the molar ratio of hydrophilic polymer to hydrophobic polymer chains on the metallic nanoparticle can be any suitable ratio, ranging from 1:10 to 10:1. In some embodiments, the ratio of hydrophilic polymer to hydrophobic polymer chain ranges from 1:5 to 5:1 (e.g., 1:1 to 1:5, 1:1 to 1:4, 1:1 to 1:3, or 1.1 to 1:2.).

The hydrophilic and hydrophobic polymers can be covalently bound to the metallic nanoparticle by any suitable method, including a chemisorption method, such as a grafting-to or a grafting-from method. In a preferred aspect of the invention, a grafting-to method is used to covalently bond the hydrophilic and hydrophobic polymers to the metallic nanoparticle. Such method typically includes contacting pre-formned, functionalized polymer(s) with a nanoparticle surface that includes one or more types of functional groups that can chemically react (e.g., form a covalent bond) with the functional groups on the polymer(s). The polymers can be used in solution or in melt form. In a grafting-from method, a monomer typically is polymerized in situ in the presence of an initiator functionalized surface of a metallic nanoparticle.

If necessary, the hydrophilic polymer and/or hydrophobic polymer can be chemically modified to provide a reactive functional group capable of forming a covalent bond with a functional group that is on the surface of the metallic nanoparticle. The surface of the metallic nanoparticle can be similarly modified, if necessary, to provide an appropriate functional group. The functional group can be, for example, amino, ammonium, hydroxyl, mercapto (—SH), sulfone (e.g., —RSO₂R′), sulfinic acid (e.g., —RSO(OH)), sulfonic acid (e.g., —RSO₂(OH)), thiocyanate, thione, thial (e.g., —C(S)H or —RC(S)H), carboxyl, halocarboxy (e.g., —OC(O)X), halo, imido, anhydrido, alkenyl, alkynyl, phenyl, benzyl, carbonyl, formyl, haloformyl (e.g., —RC(O)X), carbonato, ester (e.g., —C(O)OR), alkoxy, phenoxy, hydroperoxy, peroxy, ether, glycidyl, epoxy, hemiacetal (e.g., —OCH(R)OH or —CH(OR)OH)), hemiketal (e.g., —OCRR′OH or —CR(OR′)OH), acetal (e.g., —OCHR(OR′) or —CH(OR)(OR′)), ketal (e.g., —OCRR′(OR″) or —CR(OR′)(OR″)), orthoester, orthocarbonate ester, amido (e.g., —C(O)NRR′ or —NRC(O)R′), imino, imido, azido, azo, cyano, nitrato, nitrilo, nitrito, nitro, nitroso, pyridinyl, phosphinyl, phosphonic acid, phosphate, phosphoester, phosphodiester, boronic acid, boronic ester, borinic acid, borinic ester, or a combination thereof. In the foregoing examples, R, R′, and R″ are H, C₁₋₁₂ alkyl (e.g., methyl, ethyl, n-propyl, isopropyl, n-butyl, t-butyl, which includes a residue of an alkyl, such as methylene, ethylene, etc.), or C₃₋₈ cycloalkyl (e.g., cyclopropyl, cyclobutyl, cyclopentyl, cyclohexyl), and X is halo (e.g., fluoro, bromo, chloro, iodo).

The organic solvent is any suitable solvent that is immiscible with water. In any of the foregoing embodiments, the organic solvent comprises chloroform, methylene chloride, ethyl acetate, tetrahydrofuran, or any combination thereof. In some embodiments, the organic solvent comprises sorbitan monooleate and/or sorbitan monostearate. Preferably, the organic solvent comprises chloroform. In some embodiments, the organic solvent is chloroform.

The dispersing aid is any compound, typically with a polymeric structure, that enables the formation of the metallic nanoparticle vesicle. Typically a dispersing aid (e.g., plasticizer) that improves the separation of particles to avoid aggregation. For example, the dispersing aid can be polyvinyl alcohol, polyvinylpyrrolidone, poly(vinylpyrrolidone-co-vinyl acetate), hydroxypropyl cellulose, hydroxypropylmethyl cellulose, polysorbate (e.g., polyoxyethylene (20) sorbitan monolaurate (polysorbate 20), polyoxyethylene (20) sorbitan monopalmitate (polysorbate 40), polyoxyethylene (20) sorbitan monostearate (polysorbate 60), or polyoxyethylene (20) sorbitan monooleate (polysorbate 80)), or combinations thereof. In some embodiments, the dispersing aid comprises polyvinyl alcohol.

In the inventive method, the step of removing the organic solvent is not particularly limited as long as such step enables the formation of metallic nanoparticle vesicles. A suitable method includes evaporating the organic solvent, optionally under reduced pressure. The removal of the organic solvent can take place at room temperature or at a slightly elevated temperature (e.g., room temperature plus 1-50° C. or plus 1-40° C. or plus 1-30° C. or plus 1-20° C. or plus 1-15° C. or plus 1-10° C. or plus 1-5° C.), but typically removal of the solvent occurs at room temperature.

Due to the high optical absorption coefficient and ultrastrong electromagnetic field upon laser irradiation, nanocrystal assemblies exhibit enhanced photoacoustic (PA) signals and have been widely used for biomedical imaging. See, for example, Huang et al., J. Am. Chem. Soc. 2014, 136, 8307-8313; Moon et al., ACS Nano 2015, 9, 2711-2719; Wang et al., Nano Lett. 2008, 9, 2212-2217; and Mallidi et al., Nano Lett. 2009, 9, 2825-2831. For the vesicles described herein, the PA images demonstrate that plasmonic vesicles have much stronger PA signal than the corresponding nanorods at the same optical density (OD) value at 808 nm. At the same OD₈₀₈ value, the PA intensity of the vesicle is about 10 times higher than that of the corresponding nanorod illuminated with 808 nm laser. Furthermore, the vesicles show higher PA signals when irradiated with 808 nm laser than that with 671 nm laser, as the 808 nm laser matches the localized surface plasmon resonance (LSPR) peak of the vesicles.

In view of the improved PA signal, the small vesicles (e.g., 20-150 nm) of the invention, particularly those prepared by the method set forth herein, can be used for various diagnostic and/or treatment methods. In particular, the invention provides a method of imaging or treating cells in a subject by administering at least one vesicle to the cells. The imaging or treatment method typically will further include the application of an external energy source (e.g., laser, x-ray, gamma ray) that will interact with the vesicle and enable an imaging and/or therapeutic effect. The treatment method includes, for example, treating heart disease, stroke, atherosclerosis, or cancer (e.g., leukemia, melanoma, liver cancer, pancreatic cancer, lung cancer, colon cancer, brain cancer, ovarian cancer, breast cancer, prostate cancer, and renal cancer) in a subject. The imaging method is suitable for imaging or detection by x-rays, gamma rays, using absorption or induced x-ray fluorescence, computed tomography (CAT), ultrasound, magnetic resonance imaging (MRI), light, light microscopy, and electron microscopy.

In an embodiment, the invention provides a method of conducting photothermal therapy (PTT) comprising administering at least one vesicle, as described herein, to a cell, and applying an external energy source (e.g., laser, x-ray, gamma ray) to the cell that elevates the temperature to a level that induces cell death. The cell is from any suitable tissue that is to be treated, such as a cancer cell (e.g., leukemia, melanoma, liver cancer, pancreatic cancer, lung cancer, colon cancer, brain cancer, ovarian cancer, breast cancer, prostate cancer, and renal cancer), renal cells, cardiac cells, blood cells, and brain cells. In addition, the cell can be isolated (e.g., in vitro or ex vivo) or can be in a subject (in vivo).

The at least one vesicle can be delivered to the cells either directly or indirectly. Typically, the at least one vesicle will be administered injected, e.g., intravenously, intraarterially, intramuscularly, intradermally, or subcutaneously. For example, the at least one vesicle can be injected into an artery supplying tumor cells to be treated.

Once administered, the vesicles remain in the cell for an extended period of time (e.g., 1 day or more, 2 days or more, 3 days or more, 5 days or more, 1 week or more, 2 weeks or more, 3 weeks or more, or 1 month or more). Irradiating the vesicle with a suitable energy source increases the degradation rate of the vesicle. In a specific example, a gold nanorod vesicle comprising PEG and PLGA polymer brushes degraded upon laser irradiation. PLGA degraded into smaller segments, and the morphology of the vesicles was disrupted at day 5. Some individual gold nanorods were released at day 7 and most vesicles collapsed at day 9. Only single gold nanorods coated with PEG were observed at day 11.

The methods described herein comprise administering at least one vesicle described herein in the form of a pharmaceutical composition. In particular, a pharmaceutical composition comprises at least one vesicle described herein and a pharmaceutically acceptable carrier. The pharmaceutically acceptable excipients described herein, for example, vehicles, adjuvants, carriers or diluents, are well-known to those who are skilled in the art and are readily available to the public. Typically, the pharmaceutically acceptable carrier is one that is (i) chemically inert to the vesicle and/or any active compounds that are present and (ii) has no detrimental side effects or toxicity under the conditions of use.

The pharmaceutical composition can be administered as oral, sublingual, transdermal, subcutaneous, topical, absorption through epithelial or mucocutaneous linings, intravenous, intranasal, intraarterial, intramuscular, intratumoral, peritumoral, interperitoneal, intrathecal, rectal, vaginal, or aerosol formulations. In some embodiments, the pharmaceutical composition is administered intravenously.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The at least one vesicle can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations, include petroleum, animal, vegetable, and synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

The parenteral formulations typically will contain from about 0.5 to about 25% by weight of the vesicles in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

The dose administered to the subject (e.g., mammal, particularly humans and other mammals) in accordance with the present invention should be sufficient to affect the desired response. One skilled in the art will recognize that dosage will depend upon a variety of factors, including the age, condition or disease state, predisposition to disease, genetic defect or defects, and body weight of the subject. The size of the dose will also be determined by the route, timing and frequency of administration as well as the existence, nature, and extent of any adverse side-effects that might accompany the administration of a particular vesicle and the desired effect. It will be appreciated by one of skill in the art that various conditions or disease states may require prolonged treatment involving multiple administrations.

The inventive methods comprise administering an effective amount of at least one vesicle. An “effective amount” means an amount sufficient to show a meaningful benefit in a subject, e.g., providing a desired diagnostic image, promoting at least one aspect of tumor cell cytotoxicity (e.g., inhibition of growth, inhibiting survival of a cancer cell, reducing proliferation, reducing size and/or mass of a tumor (e.g., solid tumor)), or treatment, healing, prevention, delay of onset, halting, or amelioration of other relevant medical condition(s) associated with a particular cancer or disorder (e.g., treating heart disease, stroke, atherosclerosis). The meaningful benefit observed in the subject can be to any suitable degree (10% or more, 20% or more, 30% or more, 40% or more, 50% or more, 60% or more, 70% or more, 80% or more, or 90% or more). In some embodiments, one or more symptoms of the cancer and/or disorder (e.g., treating heart disease, stroke, atherosclerosis) are prevented, reduced, halted, or eliminated subsequent to administration of at least one vesicle, thereby effectively treating the cancer and/or disorder to at least some degree.

Effective amounts may vary depending upon the biological effect desired in the subject, condition to be treated, and/or the specific characteristics of the vesicle, and the individual. In this respect, any suitable dose of the vesicle can be administered to the subject (e.g., human), according to the desired end use (e.g., type of diagnostic image, type of cancer and/or disease to be treated). Various general considerations taken into account in determining the “effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th Ed., Mack Publishing Co., Easton, Pa., 1990, each of which is herein incorporated by reference.

If desired, the method of the present invention can further comprise loading at least one therapeutic agent (e.g., a hydrophilic therapeutic agent) in the interior of the vesicle. Alternatively, or in addition, one or more hydrophobic molecules, including a hydrophobic therapeutic agent, can be encapsulated within the hydrophobic polymer shell of the vesicle, due to a favorable hydrophobic-hydrophobic interaction. One type or more than one type, e.g., two, three, or more different therapeutic agents and/or hydrophobic molecules can be loaded into the vesicle's interior and/or the hydrophobic polymer shell.

Upon administration to a subject, the vesicles are internalized into cells. For a treatment method, therapeutic agent loaded in the interior of the vesicle should be released (e.g., using laser irradiation) once internalized in the cells. The at least one therapeutic agent can be any suitable compound, such as a biological molecule (e.g., protein, enzyme, peptide, amino acid, nucleotide, a DNA, RNA, antibody, antigen), antibacterial, antiviral, antifungal, antioxidant, antiinflammatory, analgesic, anticancer, antiallergic, antidiabetic, antihistamine, antihypertensive, anticonvulsant, antidepressant, cardiovascular agent, diagnostic aid, or wound healing agent.

The amino acid can be, for example, alanine, aspartic acid, cysteine hydrochloride, cystine, histidine, isoleucine, leucine, lysine, lysine acetate, lysine hydrochloride, methionine, phenylalanine, proline, serine, threonine, tryptophan, tyrosine, or valine.

The antibacterial agent can be, for example, amifloxacin, aminosalicylic acid, amoxicillin, ampicillin, bacitracin, biapenem, cefdinir, cephalexin, cinoxacin, ciprofloxacin, clofazimine, daptomycin, dipyrithione, dirithromycin, doxycycline, erythromycin, fosfomycin, gentamicin sulfate, lomefloxacin, nebramycin, oxacillin sodium, penicillin g benzathine, penicillin g potassium, penicillin g procaine, penicillin g sodium, penicillin v, penicillin v benzathine, penicillin v hydrabamine, penicillin v potassium, streptomycin sulfate; sulfabenzamide, tetracycline, tobramycin, or zorbamycin.

The anticonvulsant includes, for example albutoin, carbamazepine, clonazepam, ethosuximide, fluzinamide, gabapentin, magnesium sulfate, nabazenil, nafimidone hydrochloride, phenobarbital sodium, phensuximide; phenytoin; phenytoin sodium; primidone; progabide; ralitoline; thiopental sodium, valproate sodium, and valproic acid.

Examples of the antidepressant include, for example, amitriptyline hydrochloride, amoxapine, bupropion hydrochloride, cidoxepin hydrochloride, clodazon hydrochloride, dapoxetine hydrochloride, desipramine hydrochloride, dioxadrol hydrochloride, fenmetozole hydrochloride, fluotracen hydrochloride, fluparoxan hydrochloride, indeloxazine hydrochloride, ketipramine fumarate, mirtazapine; moclobemide, modaline sulfate, nisoxetine, nitrafudam hydrochloride, oxaprotiline hydrochloride, oxypertine, phenelzine sulfate, protriptyline hydrochloride, quipazine maleate, rolicyprine, sertraline hydrochloride, tampramine fumarate, trazodone hydrochloride, trebenzomine hydrochloride, trimipramine; viloxazine hydrochloride, and zometapine.

Examples of the analgesic include, for example, aspirin, acetaminophen, bicifadine hydrochloride, codeine, doxpicomine, flunixin, flupirtine maleate, flurbiprofen, ibuprofen, indoprofen, ketazocine, ketorfanol, ketorolac, naproxen, oxycodone, profadol, tradmadol veradoline hydrochloride, and xorphanol mesylate.

The antiallergic agent can be, for example, amlexanox, astemizole, azelastine hydrochloride, nedocromil, nivimedone sodium, pemirolast potassium, pirquinozol; proxicromil; repiriniast, tetrazolast meglumine, thiazinamium chloride, tiacrilast, or ortixanox.

The antidiabetic agent includes, for example, bufonnrmin, butoxamine hydrochloride; ciglitazone, etoformin hydrochloride, gliflumide, glipizide, glucagon, insulin; linogliride, metformin, palmoxirate sodium, pioglitazone hydrochloride, pirogliride tartrate, seglitide acetate, tolazamide, tolbutamide, and troglitazone.

Examples of the antifungal agent include, for example, amphotericin b, azaconazole, bifonazole, bispyrithione magsulfex, butoconazole nitrate, candicidin, ciclopirox, cisconazole, clotrimazole, dipyrithione, doconazole, fenticonazole nitrate, fluconazole, flucytosine, fungimycin, isoconazole, itraconazole, ketoconazole, naftifine hydrochloride, neomycin undecylenate, nystatin, octanoic acid, oxiconazole nitrate, pyrithione zinc, pyrrolnitrin, selenium sulfide, sulconazole nitrate, terbinafine, terconazole, tioconazole, triacetin, triafungin, undecylenic acid, and zinoconazole hydrochloride.

Examples of the antioxidant include, for example, vitamin a, retinal, 3,4-didehydroretinal, alpha-carotene, beta-carotene (beta, beta-carotene), gamma-carotene, delta-carotene, vitamin c (d-ascorbic acid, 1-ascorbic acid), and vitamin e (alpha-tocopherol), 3,4-dihydro-2,5,7,8-tetra-methyl-2-(4,8,12-trimethyltri-decyl)-2h-1-benzopyran-6-ol), beta-tocopherol, gamma-tocopherol, delta-tocopherol, tocoquinone, and tocotrienol.

Examples of anti-cancer agents include platinum compounds (e.g., cisplatin, carboplatin, oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, bendamustine), antitumor antibiotics (e.g., daunorubicin, doxorubicin, idarubicin, epirubicin, mitoxantrone, bleomycin, mytomycin C, plicamycin, dactinomycin), taxanes (e.g., paclitaxel and docetaxel), antimetabolites (e.g., 5-fluorouracil, cytarabine, premetrexed, thioguanine, floxuridine, capecitabine, and methotrexate), nucleoside analogues (e.g., fludarabine, clofarabine, cladribine, pentostatin, nelarabine), topoisomerase inhibitors (e.g., topotecan and irinotecan), hypomethylating agents (e.g., azacitidine and decitabine), proteosome inhibitors (e.g., bortezomib), epipodophyllotoxins (e.g., etoposide and teniposide), a DNA synthesis inhibitors (e.g., hydroxyurea), vinca alkaloids (e.g., vicristine, vindesine, vinorelbine, and vinblastine), tyrosine kinase inhibitors (e.g., imatinib, dasatinib, nilotinib, sorafenib, sunitinib), monoclonal antibodies (e.g., rituximab, cetuximab, panetumumab, tositumomab, trastuzumab, alemtuzumab, gemtuzumab ozogamicin, bevacizumab), nitrosoureas (e.g., carmustine, fotemustine, and lomustine), enzymes (e.g., L-Asparaginase), biological agents (e.g., interferons and interleukins), hexamethylmelamine, mitotane, angiogenesis inhibitors (e.g., thalidomide, lenalidomide), steroids (e.g., prednisone, dexamethasone, and prednisolone), hormonal agents (e.g., tamoxifen, raloxifene, leuprolide, bicaluatmide, granisetron, flutamide), aromatase inhibitors (e.g., letrozole and anastrozole), arsenic trioxide, tretinoin, nonselective cyclooxygenase inhibitors (e.g., nonsteroidal anti-inflammatory agents, salicylates, aspirin, piroxicam, ibuprofen, indomethacin, naprosyn, diclofenac, tolmetin, ketoprofen, nabumetone, oxaprozin), selective cyclooxygenase-2 (COX-2) inhibitors, or any combination thereof.

The antihistamine agent can be, for example, acrivastine, azatadine maleate, carbinoxamine maleate, cetirizine hydrochloride, clemastine, cyclizine, dexbrompheniramine maleate, diphenhydramine citrate, diphenhydramine hydrochloride, levocabastine hydrochloride, pyrabrom, temelastine, terfenadine, tripelennamine citrate, and zolamine hydrochloride.

The antihypertensive agent can be, for example, amlodipine besylate, amlodipine maleate, bemitradine, betaxolol hydrochloride, bethanidine sulfate, bupicomide, carvedilol, clonidine, diltiazem hydrochloride, diltiazem malate, fenoldopam mesylate, hydralazine hydrochloride, indacrinone, lofexidine hydrochloride, methalthiazide, metoprolol fumarate, nebivolol, pazoxide, pelanserin hydrochloride, quinapril hydrochloride, quinaprilat, ramipril, reserpine, saprisartan potassium, sodium nitroprusside, terazosin hydrochloride, tiamenidine, trimethaphan camsylate, trimoxamine hydrochloride, and zofenoprilat arginine.

The antiinflammatory agent can be, for example, alclofenae, anirolac, bromelains, budesonide, carprofen, cliprofen; cortodoxone, dexamethasone dipropionate, diclofenac potassium, diclofenac sodium, diflunisal, enolicam sodium, epirizole, etodolac, fenbufen, fenclofenac, fluazacort, flumizole, flunisolide acetate, flurbiprofen, fluretofen, fluticasone propionate, ibufenac, ibuprofen, indomethacin, indoprofen, indoxole, ketoprofen, lofemizole hydrochloride, lomoxicam, naproxen, oxaprozin, phenbutazone sodium glycerate, pirprofen, prodolic acid, seelzone, sermetacin, sudoxicam, sulinldac, tenidap, tiopinac, triclonide; triflumidate, zidometacin, and zomepirac sodium.

Examples of the antiviral agent include, for example, acyclovir, acyclovir sodium, amantadine hydrochloride, cytarabine hydrochloride, desciclovir, edoxudine, famciclovir, fialuridine, fosfonet sodium, idoxuridine, kethoxal, lamivudine, lobucavir, penciclovir, pirodavir, rimantadine hydrochloride, somantadine hydrochloride, stavudine, tilorone hydrochloride, vidarabine, viroxime, zalcitabine, and zidovudine.

Examples of the cardiovascular agent include, for example, dopexamine, and dopexamine hydrochloride.

The diagnostic aid can be, for example, arginine, butedronate tetrasodium, butilfenin, diatrizoate meglumine, diatrizoate sodium, diphtheria toxin for schick test, disofenin, etifenin, ferumoxides, ferumoxsil, fluorescein, fluorescein sodium, histoplasmin, impromidine hydrochloride, indocyanine green, iobenzamic acid, iocarmic acid, iocetamic acid, iodoxamate meglumine, iopydone, ioxilan, ioxotrizoic acid, mebrofenin, meglumine, metrizamide, pentetic acid, propyliodone, quinaldine blue, schick test control, stannous pyrophosphate, stannous sulfur colloid, tetrofosmin, tolbutamide sodium, tuberculin, and tyropanoate sodium.

The wound healing agent can be, for example, ersofermin.

For purposes of the present invention, the term “subject” preferably is directed to a mammal. Mammals include, but are not limited to, the order Rodentia, such as mice, and the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simioids (monkeys) or of the order Anthropoids (humans and apes). An especially preferred mammal is the human.

The invention is further illustrated by the following embodiments.

(1) A method of producing a vesicle comprising a metallic nanoparticle that is covalently bound to at least one hydrophilic polymer and at least one hydrophobic polymer, wherein the method comprises (i) dispersing the polymer-bound metallic nanoparticle in an organic solvent, (ii) adding an aqueous solution comprising a dispersing aid to form a mixture, (iii) sonicating the mixture to form an emulsion; and (iv) removing the organic solvent from the emulsion until the vesicle forms, wherein the polymer-bound metallic nanoparticle comprises a metallic nanoparticle that is covalently bound to at least one hydrophilic polymer and at least one hydrophobic polymer, and the vesicle has a diameter of 20-150 nm.

(2) The method of embodiment (1), wherein the metallic nanoparticle comprises gold, iron oxide, copper disulfide silver, nickel, cobalt, platinum, palladium, iridium, or mixtures thereof.

(3) The method of embodiment (2), wherein the metallic nanoparticle comprises gold.

(4) The method of any one of embodiments (1)-(3), where in the metallic nanoparticle is a quantum dot or nanorod.

(5) The method of any one of embodiments (1)-(4), wherein the hydrophilic polymer comprises at least one polymer selected from polyethylene glycol (PEG), poly(vinyl alcohol) (PVA), poly(vinylpyrrolidone) (PVP), polyacrylic acid, poly(meth)acrylic acid, polyethylenimine (PEI), poly(methyl vinyl ether), poly(styrene-maleic acid), polyethylene glycol ether, polyamide, polyacrylamide, a polypeptide, and a DNA.

(6) The method of any one of embodiments (1)-(5), wherein the hydrophilic polymer comprises polyethylene glycol (PEG).

(7) The method of any one of embodiments (1)-(6), wherein the hydrophobic polymer comprises at least one polymer selected from poly(lactic-glycoacid) (PLGA), polylactide (PLA), polystyrene, polyethylene, polypropylene, poly(2-dimethylaminoethylmethacrylate) (PDMAEMA), poly(N-isopropylacrylamide) (PNIPAM), polybutadiene, polyisoprene, poly(styrene-butadiene), polyvinyl chloride, polytetrafluoroethylene, polydimethylsiloxane, polycaprolactone, poly(4-vinylpyridine), poly(ethyl acrylate), poly(methyl acrylate), and poly(methyl methacrylate) (PMMA).

(8) The method of any one of embodiments (1)-(7), wherein the hydrophobic polymer comprises poly(lactic-glycoacid) (PLGA), polylactide (PLA), or both.

(9) The method of any one embodiments (1)-(8), wherein the organic solvent comprises chloroform, methylene chloride, ethyl acetate, tetrahydrofuran, sorbitan monooleate, sorbitan monostearate, or a combination thereof.

(10) The method of any one of embodiments (1)-(9), wherein the organic solvent comprises chloroform.

(11) The method of any one of embodiments (1)-(10), wherein the dispersing aid is selected from the group consisting of polyvinyl alcohol, polyvinylpyrrolidone, poly(vinylpyrrolidone-co-vinyl acetate), hydroxypropyl cellulose, hydroxypropylmethyl cellulose, polysorbate, and combinations thereof.

(12) The method of any one of embodiments (1)-(11), wherein the dispersing aid is polyvinyl alcohol.

(13) The method of any one of embodiments (1)-(12), wherein removing the organic solvent takes place at room temperature.

(14) The method of any one of embodiments (1)-(13), further comprising loading at least one therapeutic agent in the interior of the vesicle.

(15) A vesicle prepared by a method of any one of embodiments (1)-(14).

(16) A vesicle comprising a polymer-bound metallic nanoparticle comprising a metallic nanoparticle that is covalently bound to at least one hydrophilic polymer and at least one hydrophobic polymer, wherein the vesicle has a diameter of 20-150 nm.

(17) The vesicle of embodiment (16), wherein the metallic nanoparticle comprises gold, iron oxide, copper disulfide silver, nickel, cobalt, platinum, palladium, iridium, or mixtures thereof.

(18) The vesicle of embodiment (17), wherein the metallic nanoparticle comprises gold.

(19) The vesicle of any one of embodiments (16)-(18), where in the metallic nanoparticle is a quatum dot or nanorod.

(20) The vesicle of any one of embodiments (16)-(19), wherein the hydrophilic polymer comprises at least one polymer selected from polyethylene glycol (PEG), poly(vinyl alcohol) (PVA), poly(vinylpyrrolidone) (PVP), polyacrylic acid, poly(meth)acrylic acid, polyethylenimine (PEI), a polypeptide, and a DNA.

(21) The vesicle of any one of embodiments (16)-(20), wherein the hydrophilic polymer comprises polyethylene glycol (PEG).

(22) The vesicle of any one of embodiments (16)-(21), wherein the hydrophobic polymer comprises at least one polymer selected from poly(lactic-glycoacid) (PLGA), polylactide (PLA), poly(2-dimethylaminoethylmethacrylate) (PDMAEMA), poly(N-isopropylacrylamide) (PNIPAM), polystyrene, polycaprolactone, poly(4-vinylpyridine), and poly(methyl methacrylate) (PMMA).

(23) The vesicle of any one of embodiments (16)-(22), wherein the hydrophobic polymer comprises poly(lactic-glycoacid) (PLGA), polylactide (PLA), or both.

(24) The vesicle of any one of embodiments (16)-(23), further comprising loading at least one therapeutic agent in the interior of the vesicle.

(25) A pharmaceutical composition comprising at least one vesicle of any one of embodiments (15)-(24) and a pharmaceutically acceptable carrier.

(26) A method of conducting photothermal therapy (PTT) comprising administering at least one vesicle of any one of embodiments (15)-(24) to a cell, and applying an external energy source to the cell that elevates the temperature to a level that induces cell death.

(27) The method of embodiment (26), wherein the cell is a cancer cell.

(28) The method of embodiment (27), wherein the cancer cell is selected from leukemia, melanoma, liver cancer, pancreatic cancer, lung cancer, colon cancer, brain cancer, ovarian cancer, breast cancer, prostate cancer, and renal cancer.

The following examples further illustrate the invention but, of course, should not be construed as in any way limiting its scope.

### EXAMPLES

2-Hydroxyethyl disulphide, methoxy-poly(ethylene glycol)-thiol (MPEG-SH) with a molecular weight of 5 kDa, polyvinyl alcohol (PVA, MW 9,000-10,000), and hydrazine hydrate (50-60%) were purchased from Sigma-Aldrich (St. Louis, Mo.). Hydrogen tetrachloroaurate(III) trihydrate (HAuCl₄.3H₂O) was from Alfa Aesar (Haverhill, Mass.). Radiometal [⁶⁴Cu] was produced by the positron emission tomography (PET) department, NIH Clinical Center. All solvents unless specified were obtained from Sigma-Aldrich (St. Louis, Mo.) and used as received.

Transmission Electron Microscopy (TEM) was conducted on a Jeol JEM 2010 (Peabody, Mass.) electron microscope at an acceleration voltage of 300 kV. Scanning electron microscopy images were obtained on a Hitachi SU-70 Schottky field emission gun Scanning Electron Microscope (FEG-SEM) (Tokyo, Japan). UV-vis absorption spectra were recorded by using a Shimadzu UV-2501 spectrophotometer (Kyoto, Japan). ¹H NMR spectra were obtained on a Bruker AV300 scanner (Billerica, Mass.) using CDCl₃ as the solvent. Gel permeation chromatography (GPC) was measured on a Shimadzu HPLC system (Kyoto, Japan) using chloroform as the eluent, and the molecular weight was calibrated with polystyrene standards. Dark-field images of live cells were carried out on an Olympus71 inverted microscope (Center Valley, Pa.) with an oil-immersion dark field condenser at 100× magnification, and fluorescence images were collected using a Photometrics CoolSNAP-cf cooled CCD camera (Tucson, Ariz.). Thermogravimetric analysis (TGA) was performed on a Perkin-Elmer Diamond TG/DTA (Waltham, Mass.). Samples were placed in platinum sample pans and heated under a nitrogen atmosphere at a rate of 10° C./min to 100° C. and held for 40 min to completely remove residual solvent. Samples were then heated to 700° C. at a rate of 10° C./min.

### Example 1

This example demonstrates the synthesis of small gold nanorods.

Small gold nanorods were synthesized using a one-pot seedless method. Briefly, gold(III) chloride trihydrate (HAuCl₄ 3H₂O) (5.0 mL, 1.0 mM) was added to 5 mL of cetyltrimethylammonium bromide (CTAB) aqueous solution (0.2 M) under vigorous stirring at 30° C., followed by adding 300 μL of AgNO₃ (4.0 mM). Then 12.0 μL of HCl (37%) was rapidly added to the solution to obtain a pH of ˜11. Afterwards, 75 μL of ascorbic acid (85.8 mM) was added to the mixed solution. After the solution became clear, 7.5 μL of NaBH₄ (0.01 M) was immediately injected into the solution. After growing for 5 h, the AuNR@CTAB was purified three times by centrifugation (9000 g, 30 min).

### Example 2

This example demonstrates the synthesis of thiolated PLGA (SH-PLGA).

For the synthesis of SH-PLGA, 1.5 g of lactic acid (LA), 1.2 g of glycolic acid (GA) and 0.045 g of 2-hydroxyethyl disulfide were added into a flask with nitrogen for 30 min. Then 10 μL of tin(II) 2-ethylhexanoate (SnOct, ˜95%) was added and again flushed with nitrogen for 10 min. The polymerization of LA and GA was carried out at 130° C. for 30 h. The resulting mixture was cooled to room temperature, dissolved in tetrahydrofuran (THF) (10 mL) and precipitated into cold hexane three times and dried under vacuum to obtain the product as a white solid. To reduce the disulfide bond, 1.5 g of the as-prepared PLGA-S—S-PLGA was first dissolved in 10.0 mL of THF at 25° C. and 200 μL of tributyl phosphine was charged as the reduction catalyst. Subsequently, this reaction mixture was stirred for 30 min. The purification of the polymer (SH-PLGA) was the same as the procedure mentioned above.

### Example 3

This example demonstrates the synthesis of amphiphilic gold nanorods coated with PEG and PLGA.

To prepare amphiphilic gold nanorods attached with PEG and PLGA, 20 mL of AuNR@CTAB (50 nM) was mixed with 0.1 mL of 2-(2-aminoethoxy)ethanol and the mixture was stirred for 24 h. The modified AuNRs were purified by centrifugation at 9000 g for 10 min and further dispersed in 5 mL of DMSO. Amphiphilic AuNR@PEG/PLGA was synthesized by a “grafting to” reaction. Briefly, the mixture of 10 mg of thiolated PEG (PEG-SH, Mₙ=5000 g/mol) and 12 mg of thiolated PLGA (PLGA-SH, Mₙ=8000 g/mol) was slowly added into the modified AuNR dispersion, and the solution was stirred for 12 h. The AuNR@PEG/PLGA was purified by centrifugation (10,000 g, 15 min) and dispersed in 5 mL of chloroform.

The ratio of PEG and PLGA grafts on the gold nanorod surface were calculated as follows. ¹H-NMR measurement shows that the resonance of —CH₂—CH₂—O— (3.65 ppm) of PEG and that of —CO—CH₂—O— group (1.54 ppm) of PLGA has a ratio of 4:3, which leads to a molar ratio of 2:3 for ethylene glycol (EG) and LGA monomer. With the molecular weights of PEG (Mₙ=5 KDa) and PLGA (Mₙ=8 kDa), the ratio of PEG and PLA grafts can be calculated using Equation S1, where M_{nLGA} is the molecular weight of LGA monomer. Because of the ratio of LA to GA is 1:1, thus the molecular weight of LGA is: Mn_{LGA}=0.5 Mn_{LA}+Mn_{GA}. Mn_{EG} is the molecular weight of EG monomer. The PEG to PLGA ratio is thus 1:1.2 (PEG:PLGA).

$$Ratio ( PEG \text{:} PLGA ) = Ratio ( EG \text{:} LGA ) ( \frac{{Mn}_{PLGA} / {Mn}_{LGA}}{{Mn}_{PEG} / {Mn}_{EG}} ) \qquad ( Equation S 1 )$$

The PEG/PLGA graft density was calculated from the TGA data as follows. Given the size of a gold atom (0.0125 nm³), the number of gold atom (N_{Au atom}) in a gold nanorod (˜8×2 nm) can be calculated using Equation S2, where r is the radius and L is the length of the gold nanorods. There were 11,966 gold atoms per small nanorod and therefore the molar mass (M_{Au nanorod}) of the gold nanorods was 197 N_{Au atom}. Combining the molar mass of the gold nanorod, the ratio of PEG and PLGA and the weight fraction obtained in TGA analysis, the average number of polymer grafts can be calculated by Equation S3, where W_{polymer} is the weight fraction (33%) of the organic part, W_{Au nanorod} is the weight fraction of gold nanorod and M_{PEG+1.2PLGA} is the sum of the molar mass of 1 PEG and 1.2 PLGA grafts. Therefore there were 22 grafts per nanorod, which include 10 PEG chains and 12 PLGA chains, and the graft density was ˜0.38 chains/nm²

$$\begin{matrix}{N}_{Au atom} = \frac{{V}_{Au nanorod}}{{V}_{Au atom}} = ( \frac{π {r}^{2} L}{{V}_{Au atom}} ) & ( Equation S 2 ) \\ {N}_{grafts per nanorod} = ( \frac{{2.2}_{polymer} / {Mn}_{PEG + 1.2 PLGA}}{{W}_{Au nanorod} / {M}_{Au nanorod}} ) & ( Equation S 3 )\end{matrix}$$

### Example 4

This example demonstrates the synthesis of ultrasmall AuNR@PEG/PLGA vesicles in an embodiment of the invention. See FIG. 1.

AuNR@PEG/PLGA (5 mg) was first dissolved in 800 μL of chloroform. To prepare the aqueous phase for microemulsion, 80 mg of polyvinyl alcohol (PVA, MW 9,000-10,000 g/mol) as a polymer stabilizer was dissolved in 8 ml of D.I. water at 60° C. After PVA was completely dissolved, the clear solution was cooled to room temperature. The organic phase was added to the PVA solution and emulsified for several minutes by pulsed sonication (100 watts and 22.5 kHz, MISONIX ultrasonic liquid processors, XL-2000 series, Farmingdale, N.Y.). The oil-in-water emulsion droplets were then stirred at room temperature for 24 h to evaporate the chloroform. The resulting AuNR@PEG/PLGA vesicles were washed with D.I. water 3 times to remove excess PVA.

In the vesicle, PLGA forms the vesicular shell embedded with AuNRs and PEG extends to both the inner and outer sides of the vesicular shell to stabilize the vesicles in aqueous solution and prevent aggregation under physiological condition. The dynamic light scattering (DLS) results show the size and polydispersity index of the as-prepared vesicle as 60 nm and 0.16, respectively. It was determined that the size of the vesicle increases with increased concentration of AuNR@PEG/PLGA in the initial stock solution, and the volume ratio of chloroform to water. In comparison with AuNRs, vesicles show significant red-shifts of both the longitudinal and transverse LSPRs of AuNRs due to the strong plasmonic coupling of the nanorods in the vesicular shell (Halas et al., Chem. Rev. 2011, 111, 3913-3961). As shown in FIG. 2, different sized vesicles have peak absorbance between 800-1050 nm. The LSPR peaks of larger vesicles shift towards longer wavelengths. The reason is that the larger the vesicle become, the more important are the higher-order modes as the light can no longer polarize the nanovesicle homogeneously, which is a retardation effect. These higher-order modes peak at lower energies and therefore the UV-vis spectra red shifts with increasing vesicle size. The 60 nm vesicles have peak absorbance around 800 nm, which is suitable for irradiation by 808 nm laser.

### Example 5

This example demonstrates the synthesis of AuNR@PEG/PS vesicles in an embodiment of the invention.

To prepare amphiphilic gold nanorods coated with PEG and poly(styrene) (PS), thiolated PS (SH-PS) was first synthesized. Briefly, 2 mL anisole solution of 30 mg 2, 2′-dithiobis [1-(2-bromo-2-methyl-propionyloxy)] ethane (DTBE), 1.3 g styrene, and 35 μL PMDETA were mixed in a flask and flushed with nitrogen for 30 min. Then 23 mg CuBr was added, and the reaction mixture was again flushed with nitrogen for 10 min. The mixture was stirred for 12 h at 110° C. The resulting mixture was cooled to room temperature, dissolved in tetrahydrofuran (THF) (20 mL), precipitated into cold ethanol three times, and dried under vacuum to obtain the product as a white solid. To reduce the disulfide bond, 1 g of the as-prepared PS—S—S-PS was first dissolved in 15.0 mL of THF at 25° C. and 150 μL of tributyl phosphine was charged as the reduction catalyst. Subsequently, this reaction mixture was stirred for 30 min. The purification of the polymer (SH-PS) was the same as the procedure mentioned above. Yield: 86%, Mₙ=8300 g/mol.

To prepare amphiphilic AuNR@PEG/PS, 30 mL of AuNR@CTAB (50 nM) was mixed with 0.15 mL of 2-(2-aminoethoxy)ethanol, and the mixture was stirred for 24 h. The modified AuNRs were purified by centrifugation at 9000 g for 10 min and further dispersed in 8 mL of DMSO. The mixture of 10 mg of thiolated PEG (PEG-SH, Mₙ=5000 g/mol) and 12 mg of thiolated PS (SH-PS, Mₙ=8300 g/mol) was slowly added into the modified AuNR dispersion, and the solution was stirred for 12 h. The AuNR@PEG/PS was purified by centrifugation (10,000 g, 15 min) and dispersed in 5 mL of chloroform. The AuNR@PEG/PS vesicle was prepared using the method described above.

### Example 6

This example demonstrates the NIR laser irradiation of a AuNR@PEG/PLGA vesicle and the calculation of the photothermal conversion efficiency.

A total of 500 μL small AuNR@PEG/PLGA vesicles (60 nm) or small AuNRs based on the same concentration of AuNR of 0.05 nM in 1 mL Eppendorf vial (Hamburg, Germany) was irradiated with a 808 nm diode laser (spot size: 1 cm) at a power density of 0.4 or 0.8 W/cm² for 5 min, respectively. Real-time thermographic images and temperature elevation of the vesicle aqueous solution were taken by an infrared thermographic camera as a function of irradiation time. Phosphate-buffered saline (PBS) was selected as a negative control.

The temperature of the vesicle solution (0.1 nM AuNRs) rapidly reached 75.2° C. after irradiation with the laser (0.8 W/cm² for 5 min). Treatment at 0.4 W/cm² for 5 min still allowed the temperature of the vesicle solution to increase to 62.6° C. However, the AuNR solution with the same concentration irradiated with 0.8 W/cm² laser showed only a modest temperature increase (43.5° C.).

The photothermal conversion efficiency (η) of the vesicle and AuNR were calculated according to the energy balance of the system as follows:

η=(hSΔTₘₐₓ−Qₛ)/I(1−10^{-A808}) (Equation S4)

τₛ=m_{D}C_{D}/hS (Equation S5)

in which h is the heat-transfer coefficient, S is the surface area of the container, ΔDTₘₐₓ is the temperature change of the vesicle solution at the maximum steady-state temperature, I is the laser power, A808 is the absorbance of the BGVs at 808 nm, and Qₛ is the heat associated with light absorption by the solvent. The variable is is the sample-system time constant, and m_{D} and C_{D} are the mass (0.2 g) and heat capacity (4.2 J/g) of the deionized water used as the solvent. According to Equations S4 and S5, the η value of the small AuNR vesicle was determined to be 51%. The η of the small AuNR was 23% based on the same calculation method. Thus, the η value of the vesicles is about two-fold higher than AuNRs. The matching of the LSPR peak of vesicle with the laser and strong plasmonic coupling of the AuNRs in the vesicular shell contribute to the much better photothermal conversion efficiency of the vesicles over AuNRs (Huang et al., J. Am. Chem. Soc. 2014, 136, 8307-8313).

### Example 7

This example demonstrates the degradation of AuNR@PEG/PLGA vesicles.

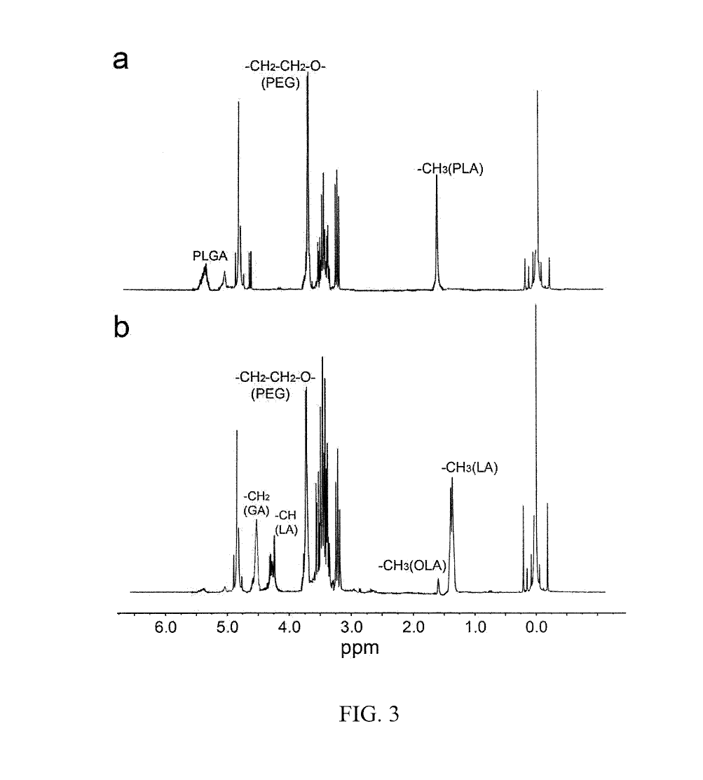

In order to allow the vesicles to degrade over time in vivo, thiolated PLGA (PLGA-SH) was synthesized with a 50:50 monomer ratio as the hydrophobic polymer brush attached onto AuNR surface to form vesicular shell. During the degradation of vesicles, change of PLGA to smaller segments is expected to change the morphology and integrity of the vesicles. As observed in a TEM image, the morphology of the vesicles was disrupted at day 5 and some individual AuNRs were released at day 7. Further incubation leads to collapse of most vesicles at day 9 and observation of only single AuNRs at day 11. See FIG. 3. Dynamic light scattering (DLS) measurements showed decreased hydrodynamic size of the vesicles with increased incubation time, consistent with the TEM results and spectral blue shift observed in UV-vis analysis. Based on ¹H-NMR results, PLGA was nearly completely degraded at day 11, and the vesicle was disrupted into single hydrophilic AuNRs coated with PEG (AuNR@PEG). Laser irradiation also led to rapid deassembly of the nanovesicles into individual AuNRs. Of note is the final small AuNR@PEG induced by degradation of vesicle still showed high solubility and stability in PBS or medium, thus facilitating clearance from the body (Otsuka et al., Adv. Drug Deliv. Rev. 2003, 55, 403-419; and Kim et al., Acec. Chem. Res. 2013, 46, 681-691).

As a control experiment, a non-dissociable vesicle assembled from AuNR coated with PEG and non-biodegradable poly(styrene) (AuNR@PEG/PS) was prepared. Both SEM images and DLS results showed that no obvious morphology and size changes of the AuNR@PEG/PS vesicle were observed after incubation in cell culture medium for 10 days.

### Example 8

This example demonstrates the synthesis of radioactive [⁶⁴Cu] labeled plasmonic vesicles.

To prepare radiometal [⁶⁴Cu] doped plasmonic vesicles, 3 μL ⁶⁴CUCl₂ was pre-mixed with 1.1 mg of Na-ascorbate (in 0.1 M borate buffer pH 8.6). Then 200 μL of vesicles (0.8 mg/mL) were added. The mixture was shaken at 37° C. for 1 h. The resulting [⁶⁴Cu] labeled vesicles were purified by centrifugation (4000 g, 5 min) three times to remove unreacted [⁶⁴Cu] and excess reagents. The purified [⁶⁴Cu] labeled vesicles were then dispersed in PBS. The labeling efficiency was determined using instant thin-layer chromatography (ITLC) plates and 0.1 M citric acid pH 5 as an eluent. Free ⁶⁴Cu elutes to the solvent front (r_{f}=0.6-0.8) and ⁶⁴Cu—AuNR vesicle stays at the origin (r_{f}=0-0.1). [⁶⁴Cu] labeled small AuNR@PEG was prepared using the same approach.

### Example 9

This example demonstrates the in vitro cytotoxicity of AuNR vesicles.

A standard Cell Counting Kit-8 (CCK-8) was utilized to analyze the cytotoxicity of AuNR vesicles following a general protocol. Briefly, U87MG cells were seeded in a 96-well plate with the concentration of 5×10⁴ cells/well. After incubation at 37° C. for 24 h, AuNR vesicles with a final concentration of 0.25, 0.5, 1 or 2 nM were incubated with cells for 2, 4, 8, 16 and 24 h, respectively, after which 10 μl of CCK-8 solution was added to each well of the 96-well plate and incubated for another 4 h. The amount of an orange formazan dye, produced by the reduction of WST-8 (active gradient in CCK-8) by dehydrogenases in live cells, is directly proportional to the quantity of live cells in the well. Therefore, by measuring the absorbance of each well at 450 nm using a microplate reader, cell viability could be determined with the calculation of the ratio of absorbance of experimental well to that of the cell control well. All experiments were triplicated and results were averaged.

### Example 10

This example demonstrates photothermal therapy of cells incubated with AuNR@PEG/PLGA vesicles and AuNR@PEG.

A standard Cell Counting Kit-8 (CCK-8) was utilized to analyze the cytotoxicity of AuNR@PEG/PLGA vesicles following a general protocol. Briefly, the U87MG human, glioma cells were seeded in a 96-well plate (1×10⁴ cells/well). After incubation at 37° C. for 24 h, AuNR@PEG/PLGA vesicles or AuNR@PEG with a final concentration of 0.5 nM of gold nanorod were added and incubated for 4 h, the cells were then washed with PBS and 100 L fresh medium was added. The cells were exposed to an 808 nm laser at 0.2, 0.4 and 0.8 W/cm² for 5 min, respectively. After incubation for another 24 h, the viability of cancer cells was examined using the standard CCK-8 assay. All experiments were triplicated and results were averaged.

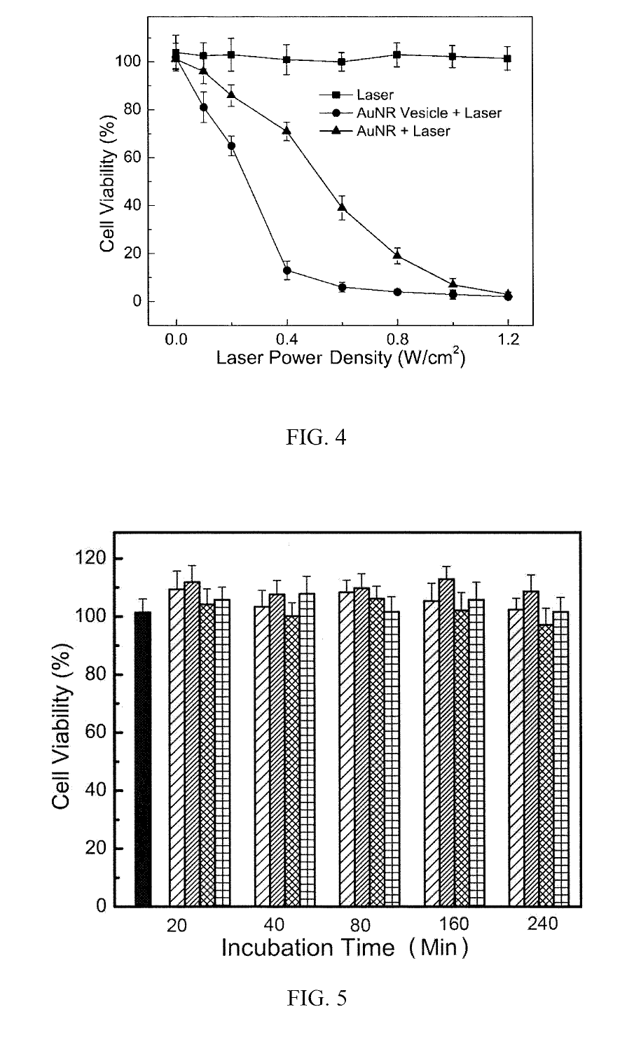

After exposure to the laser (0.8 W/cm², 5 min), all vesicle-treated cells underwent photothermal destruction within the laser spot as shown by calcein AM (live cell) and propidium iodide (dead cell) cell viability staining (FIG. 4). Exposing the cells to either vesicles or 808 nm laser alone did not affect cancer cell viability.

Treatment with laser at 0.4 W/cm² for 5 min caused over 90% cell death. In contrast, the cells incubated with vesicles for 24 h without laser irradiation had almost no cell death (FIG. 5).

### Example 11

This example demonstrates in vivo photoacoustic and positron emission tomography (PET) imaging of small AuNR@PEG/PLGA vesicles.

In vivo photoacoustic imaging using the AuNR@PEG/PLGA vesicles was carried out using the U87MG tumor xenograft model. All animal experiments were approved by the animal care and use committee (ACUC) of the National Institutes of Health Clinical Center (NIH CC). The U87MG tumor-bearing nude mice were prepared by inoculating cells (1×10⁶ cells in 100 μL PBS) into the right shoulder of each mouse (female, 7 weeks old) under anesthesia, and the tumor was allowed to grow for about 15 days, when the volume was approximately 70 mm³. The vesicle solution in PBS (200 μL, 500 μg/mL) was then injected intravenously into the tumor-bearing nude mice, and the tumor region of the mice was scanned with VisualSonic Vevo 2100 LAZR system (Toronto, Canada) equipped with a 40 MHz, 256-element linear array transducer at different time points.

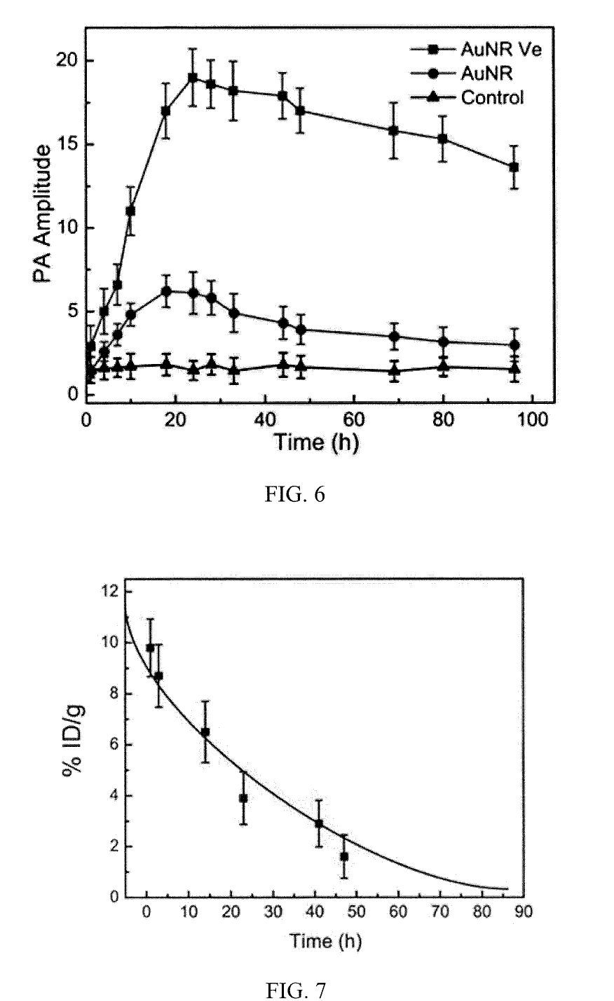

The accumulation of the vesicles in the tumor was confirmed by continuous enhancement of the 2D and 3D PA images and intensities in the tumor region over time (FIG. 6). In comparison with the AuNR vesicle, the mice treated with the same amount of small PEGylated AuNR showed much weaker PA signal in the tumor region at the same time points (FIG. 6), suggesting lower uptake of the small AuNR in tumor region and weak PA signal of the AuNR.

For in vivo PET imaging, vesicles were labeled with radio-metal [⁶⁴Cu]. When the tumor size reached ˜70 mm³, 150 μCi of [⁶⁴Cu]AuNR@PEG/PLGA vesicles were injected intravenously into each tumor mouse. PET scans and image analysis were conducted using an Inveon microPET scanner (Siemens Medical Solutions, Malvern, Pa.) at 2 h, 6 h, 24 h, and 48 h post-injection.

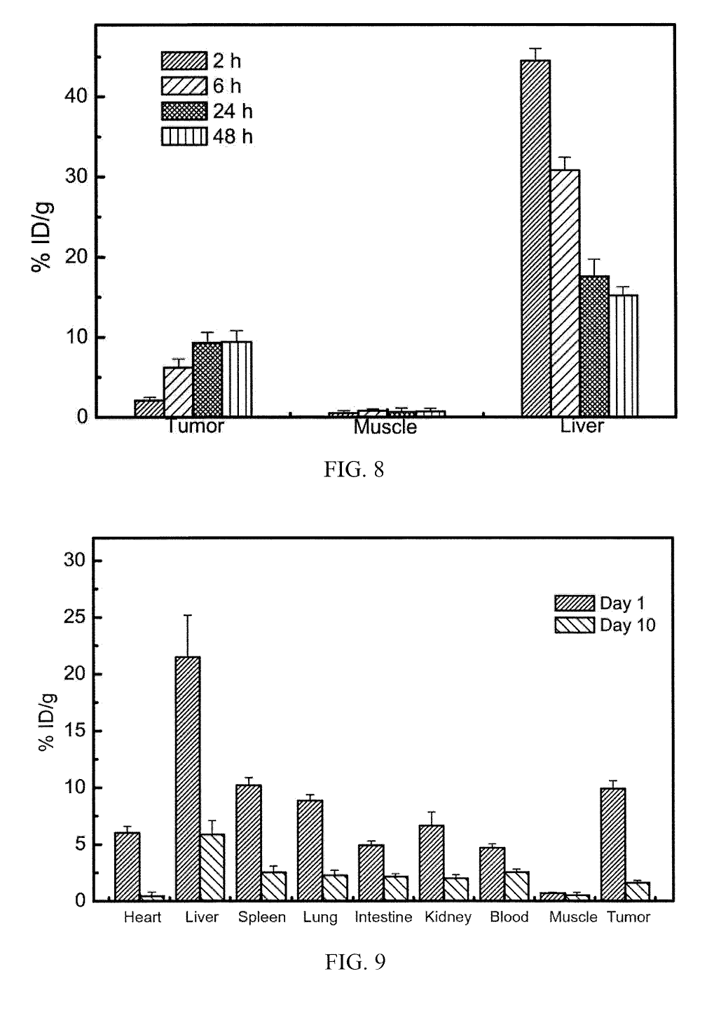

The clearance of AuNR vesicles in the blood followed a simple exponential decay curve, with a half-life of ˜18 h (FIG. 7). As shown in FIG. 8, the tumor uptake of [⁶⁴Cu]—AuNR vesicle was ˜1.8% ID/g at 2 h post-injection, which was increased to ˜4.2% ID/g at 6 h and further to ˜9.5% ID/g at 24 h (n=4/group). Efficient accumulation of AuNR vesicles in the tumor tissue was further confirmed by the ex vivo biodistribution data at 24 h time point measured by measuring the Au content of major organs and tissues via inductively coupled plasma-mass spectrometry (ICP-MS) (FIG. 9). Most of the vesicles were removed from the body at day 10 post-injection as most of the vesicles had been disassembled into single AuNR@PEG triggered by the hydrolysis of PLGA (FIG. 9), which is necessary and beneficial for further clinical translation (Hubbell et al., Science 2012, 337, 303-305; Barenholz, Nat. Nano. 2012, 7, 483-484; and Riehemann et al., Angew. Chem. Int. Ed. 2009, 48, 872-897). The AuNR@PEG are stable under physiological conditions and readily clear from the body.

As a control experiment, the tumor uptake of ⁶⁴Cu-labeled small PEGylated AuNR is 4.68 ID/g at 24 h post-injection (FIG. 10), which is about half that of AuNR vesicles, due to the rapid clearance of the small AuNR from the body and less EPR effect.

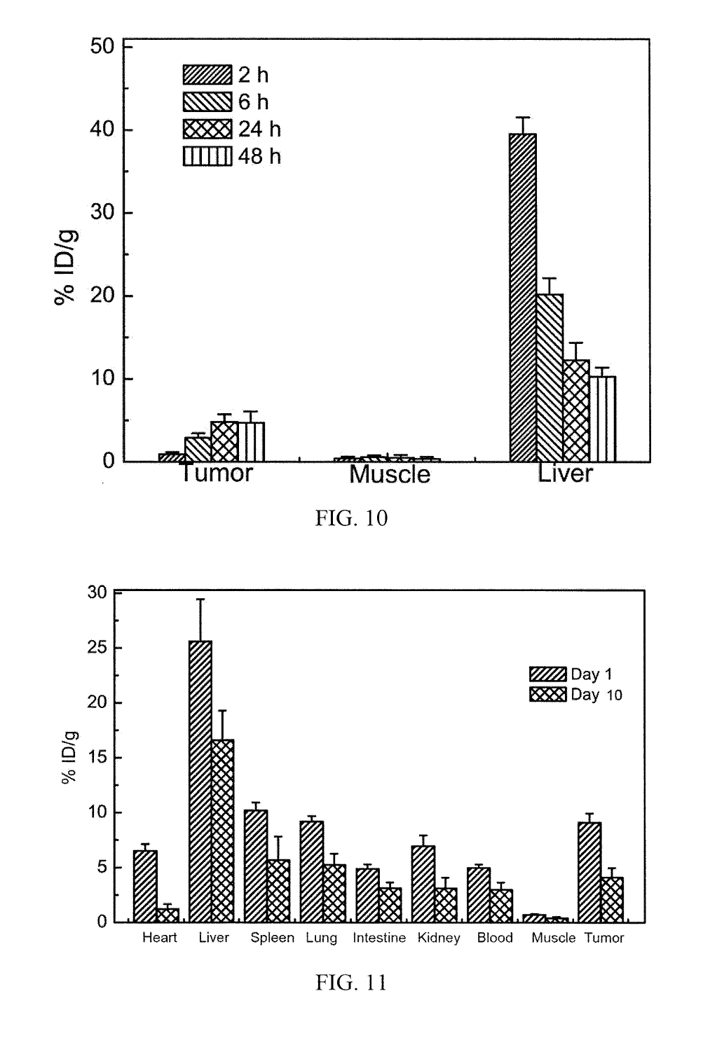

In the tumor bearing mice treated with AuNR@PEG/PS vesicles under the same conditions, most of the vesicles were retained in the body of the mice, such as the liver, which showed a slower excretion from the body than AuNR@PEG/PLGA vesicle (FIG. 11).

### Example 12

This example demonstrates in vivo photothermal cancer therapy.

When the tumor volume was approximately 70 mm³ (15 days after inoculation), an aliquot (200 μL) of AuNR vesicles (500 μg Au/mL) and PEGylated AuNRs (500 μg Au/mL) or PBS was intravenously injected into the mice under anesthesia (n=5/group). At 24 h after the injection, the entire region of the tumor was irradiated with 808 nm laser at 0.4 or 0.8 W/cm² for 5 min. During irradiation, real-time thermal images of the tumor region were acquired using a SC300 infrared camera (FLIR). The average temperature of the tumor region was analyzed using FLIR analyzer professional software. After laser irradiation, a caliper was applied to measure the dimensions of the tumor at various time points. The tumor volume V (mm³) was calculated based on the formula: V=LW²/2, where L and W refer to the length and width of tumor in millimeters.

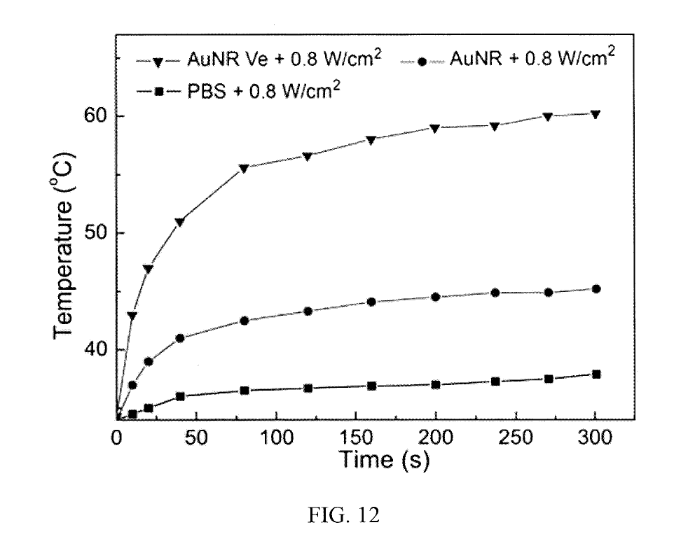

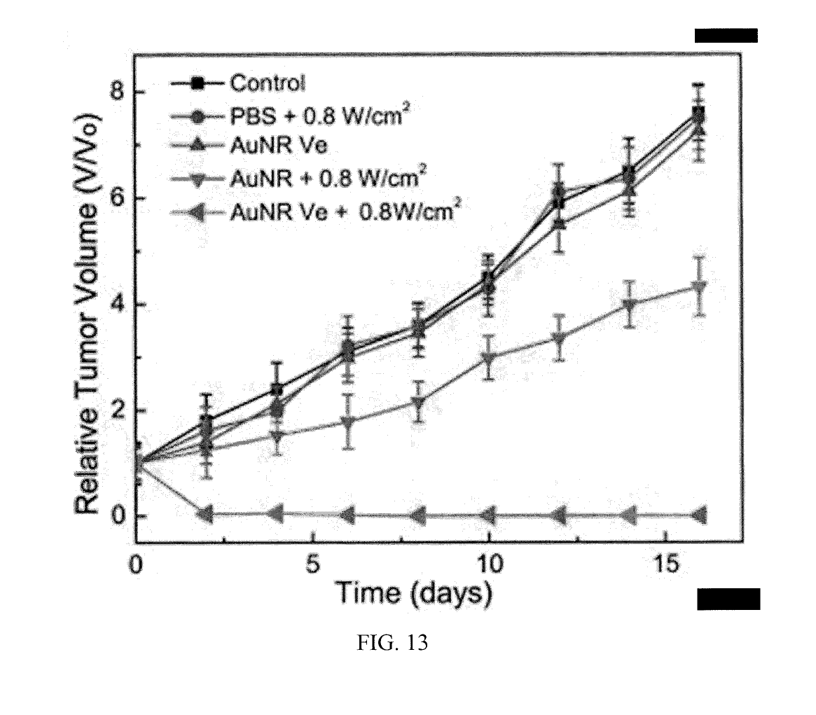

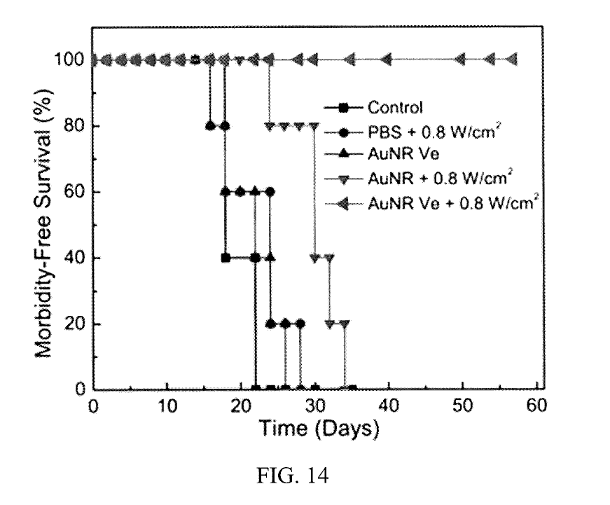

The mice treated with PBS showed negligible temperature increase after 5 min of NIR laser irradiation at power density of 0.8 W/cm² (FIG. 12). However, the mice injected with AuNR vesicles showed a tumor temperature increase of up to 20° C. after 5 min irradiation with 808 nm laser (0.8 W/cm²), which was much higher than that of small AuNRs treated mice (˜5° C. temperature increase). This therapy raised the tumor tissue temperature well above the damage threshold necessary to induce irreversible tissue damage. As shown in FIG. 13, when tumor-bearing mice treated with AuNR vesicles and 808 nm laser, all the tumors were completely ablated and no reoccurrence was observed, while tumor mice treated with small AuNRs and laser irradiation did not show complete regression of the tumors and all died within 40-50 days due to the recurrent tumors (FIG. 14).

Furthermore, no significant body weight loss was noticed after small vesicle-induced PTT treatment, indicating no acute side effects. Hematoxylin and Eosin (H&E) staining of tumor sections after laser treatment. Intensive necrosis area stained by eosin dominated tumor section in vesicle plus laser treated group. However, in the PBS or laser only treatment groups, the histological section showed infiltrating tumor cells with highly pleomorphic nuclei and many mitoses, indicating limited benefit from laser treatment alone. No obvious inflammation or damage was observed of major organs, including heart, liver, spleen, lung, and kidneys, treated with vesicles and laser on day 10, suggesting the low cytotoxicity, and biocompatibility of the vesicle.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of producing a vesicle comprising a polymer-bound metallic nanoparticle, wherein the method comprises
dispersing the polymer-bound metallic nanoparticle in an organic solvent,
adding an aqueous solution comprising a dispersing aid to form a mixture,
sonicating the mixture to form an emulsion; and
removing the organic solvent from the emulsion until the vesicle forms, wherein
the polymer-bound metallic nanoparticle comprises a metallic nanoparticle that is covalently bound to at least one hydrophilic polymer and at least one hydrophobic polymer, and
the vesicle has a diameter of 20-150 nm.

2. The method of claim 1, wherein the metallic nanoparticle comprises gold, iron oxide, copper disulfide silver, nickel, cobalt, platinum, palladium, iridium, or mixtures thereof.

3. The method of claim 2, wherein the metallic nanoparticle comprises gold.

4. The method of claim 1, where in the metallic nanoparticle is a quantum dot or nanorod.

5. The method of claim 1, wherein the hydrophilic polymer comprises at least one polymer selected from polyethylene glycol (PEG), poly(vinyl alcohol) (PVA), poly(vinylpyrrolidone) (PVP), polyacrylic acid, poly(meth)acrylic acid, polyethylenimine (PEI), poly(methyl vinyl ether), poly(styrene-maleic acid), polyethylene glycol ether, polyamide, polyacrylamide, a polypeptide, and a DNA.

6. The method of claim 1, wherein the hydrophilic polymer comprises polyethylene glycol (PEG).

7. The method of claim 1, wherein the hydrophobic polymer comprises at least one polymer selected from poly(lactic-glycoacid) (PLGA), polylactide (PLA), polystyrene, polyethylene, polypropylene, poly(2-dimethylaminoethylmethacrylate) (PDMAEMA), poly(N-isopropylacrylamide) (PNIPAM), polybutadiene, polyisoprene, poly(styrene-butadiene), polyvinyl chloride, polytetrafluoroethylene, polydimethylsiloxane, polycaprolactone, poly(4-vinylpyridine), poly(ethyl acrylate), poly(methyl acrylate), and poly(methyl methacrylate) (PMMA).

8. The method of claim 1, wherein the hydrophobic polymer comprises poly(lactic-glycoacid) (PLGA), polylactide (PLA), or both.

9. The method of claim 1, wherein the organic solvent comprises chloroform, methylene chloride, ethyl acetate, tetrahydrofuran, sorbitan monooleate, sorbitan monostearate, or a combination thereof.

10. The method of claim 1, wherein the organic solvent comprises chloroform.

11. The method of claim 1, wherein the dispersing aid is selected from the group consisting of polyvinyl alcohol, polyvinylpyrrolidone, poly(vinylpyrrolidone-co-vinyl acetate), hydroxypropyl cellulose, hydroxypropylmethyl cellulose, polysorbate, and combinations thereof.

12. The method of claim 1, wherein the dispersing aid is polyvinyl alcohol.

13. The method of claim 1, wherein removing the organic solvent takes place at room temperature.

14. The method of claim 1, further comprising loading at least one therapeutic agent in the interior of the vesicle.

15. A vesicle prepared by a method of claim 1.

16. A vesicle comprising a polymer-bound metallic nanoparticle comprising a metallic nanoparticle that is covalently bound to at least one hydrophilic polymer and at least one hydrophobic polymer, wherein the vesicle has a diameter of 20-150 nm.

17. The vesicle of claim 16, wherein the metallic nanoparticle comprises gold, iron oxide, copper disulfide silver, nickel, cobalt, platinum, palladium, iridium, or mixtures thereof.

18. The vesicle of claim 17, wherein the metallic nanoparticle comprises gold.

19. The vesicle of claim 16, wherein in the metallic nanoparticle is a quantum dot or nanorod.

20. The vesicle of claim 16, wherein the hydrophilic polymer comprises at least one polymer selected from polyethylene glycol (PEG), poly(vinyl alcohol) (PVA), poly(vinylpyrrolidone) (PVP), polyacrylic acid, poly(meth)acrylic acid, polyethylenimine (PEI), a polypeptide, and a DNA.

21. The vesicle of claim 16, wherein the hydrophilic polymer comprises polyethylene glycol (PEG).

22. The vesicle of claim 16, wherein the hydrophobic polymer comprises at least one polymer selected from poly(lactic-glycoacid) (PLGA), polylactide (PLA), poly(2-dimethylaminoethylmethacrylate) (PDMAEMA), poly(N-isopropylacrylamide) (PNIPAM), polystyrene, polycaprolactone, poly(4-vinylpyridine), and poly(methyl methacrylate) (PMMA).

23. The vesicle of claim 16, wherein the hydrophobic polymer comprises poly(lactic-glycoacid) (PLGA), polylactide (PLA), or both.

24. The vesicle of claim 16, further comprising loading at least one therapeutic agent in the interior of the vesicle.

25. A pharmaceutical composition comprising at least one vesicle of claim 16 and a pharmaceutically acceptable carrier.

26. A method of conducting photothermal therapy (PTT) comprising administering at least one vesicle of claim 16 to a cell, and applying an external energy source to the cell that elevates the temperature to a level that induces cell death.

27. The method of claim 26, wherein the cell is a cancer cell.

28. The method of claim 27, wherein the cancer cell is selected from leukemia, melanoma, liver cancer, pancreatic cancer, lung cancer, colon cancer, brain cancer, ovarian cancer, breast cancer, prostate cancer, and renal cancer.
